(19) United States
(12) Patent Application Publication
(10) Pub. No.: US 2010/0119654 A1
(43) Pub. Date: May 13, 2010

(54) METHOD FOR REDUCING THE VIRUS AND MICRO-ORGANISM CONTENT OF BIOLOGICAL EXTRACTS WHICH CONTAIN SOLIDS AND EXTRACT PRODUCED ACCORDING TO THE METHOD

(76) Inventors: Christian Rämsch, Gross Nordende (DE); Thomas Schräder, Uetersen (DE); Thomas Moest, Moorrege (DE); Manfred Kurfürat, Moorrege (DE)

Correspondence Address: FRIEDRICH KUEFFNER, 317 MADISON AVENUE, SUITE 910, NEW YORK, NY, 10017, US

(21) Appl. No.: 12/311,894

(22) PCT Filed: Aug. 7, 2008
(86) PCT No.: PCT/EP2008/006474

§ 371 Date: Jun. 30, 2009

(30) Foreign Application Priority Data
Sep. 14, 2007 (DE) 20 2007 012 915.7
Jan. 3, 2008 (DE) 20 2008 000 137.4
Mar. 18, 2008 (DE) 20 2008 003 806.5

(51) Int. Cl. A23L 1/28; C07K 14/435

(52) U.S. Cl. 426/63

(57) ABSTRACT

The invention relates to a method for reducing the virus and micro-organism content of biological extracts which contain solids. It is provided that the method comprises the steps
(a) provision of a biological extract which contains solids in the form of a suspension, which consists of a liquid phase and solid particles dispersed therein, wherein the extract in step (a) comprises a mixture of enzymes, proteins and peptides, some of which is dissolved in the liquid phase and some of which can be bound to the solid particles or is present in pulverulent, solid form; and
(b) irradiation of the biological extract provided in step (a);
   wherein
   the radiation used for the irradiation is selected from the group comprising ultra-violet radiation, x-ray radiation, β radiation and γ radiation; and
   the enzymic activity of the biological extract which contains solids after irradiation is at least 50% of the enzymic activity of the biological extract which contains solids before irradiation.

### FIELD OF APPLICATION

The invention relates to a method for reducing the virus and micro-organism content of biological extracts which contain solids, extracts produced according to the method, a biological extract which contains solids and is produced by means of this method, and uses of the product.

### PRIOR ART

Viruses are nucleic acids which are surrounded by a protein shell. In the case of enveloped viruses there is another outer lipid envelope. As viruses cannot replicate independently, they are reliant on hosts. Accordingly they occur in virtually all living things in the world. Extracts which are obtained from biological source material can sometimes have a high virus content. Very few of the known viruses are pathogenic for humans, as they have a high host specificity. In order to rule out a hazard to consumers from the start, extracts which are intended for human consumption or are used as an active agent in medicaments should in principle have no virus content or one which is as low as possible. The actual production method does not always lead to a significant inactivation or removal of the viruses present so that, in particular in the production of pharmaceutical active agents, additional depletion or inactivation steps must be integrated in the method.

Numerous methods are described for the depletion or inactivation of viruses and micro-organisms [K. H. Wallhäuser, Praxis der Sterilisation Desinfektion-Konservierung (Practice of sterilisation disinfection preservation), Thieme Verlag, Stuttgart 1995]. In addition to mechanical removal by for example chromatography or filtration, these contaminations can be inactivated selectively by adding chemical compounds. The latter method is however problematical in that these compounds must be removed again completely so that they do not cause any toxic effects in the end product. Physical methods such as heat treatment or irradiation are likewise usual methods for inactivating viruses or micro-organisms.

A particular challenge is the inactivation or removal of viruses from complex biological extracts whose active substances are enzyme mixtures, without destroying or changing the enzymic activity of the proteins.

The inactivation of micro-organisms or viruses in aqueous solutions by irradiation is a common and much described method (Oye A K and Rimstad E. 2001; Hirayama et al. 1998; Henderson et al. 1992; Chin et al. 1997). In addition to β and γ radiation, x-ray or UV radiation is also used in this case.

Of particular economic interest is the pharmaceutical active agent pancreatin, which is obtained as an extract from the pig pancreas and used in dried form as an oral therapeutic agent, as described in DE-A-3248588. The active substances in pancreatin include different enzymes which break down polymers, such as lipases, amylases and proteases. A prerequisite for the effectiveness of the therapeutic agent is that all the enzymes are present in the active agent in a certain ratio and in an active form. A particular feature of pancreatin is that the enzymes contained are present partially in solution and partially bound to particles, and it is thus a suspension.

Investigations into the virus content of pancreatin have shown that porcine parvovirus (PPV) can be found as an infectious particle as the only virus in pancreatin. The zoonotic viruses EMCV, PEV9 and HEV as well as rotavirus and reovirus could not be found, either as an infectious particle or on the genome level. In principle, pharmaceutical active agents should contain no infectious viruses. Although PPV is not pathogenic for humans according to current knowledge, the objective should be PPV-free pancreatin. As the current production process is obviously not capable of removing the existing PPV content completely (titer in pancreatin up to a maximum of 2.8 log/g), additional virus-reducing steps must be implemented.

Classical virus-inactivating methods such as dry or moist heat or virus-depleting methods such as filtration or chromatography cannot be used in most cases with extracts from biological source material and in particular with organ extracts without changes to the composition and/or high product losses. A particular problem of these extracts is often undissolved components, which give them a suspension-like property. This leads to blocking of filters or chromatography columns. Furthermore, the active substances are often distributed both in the dissolved and in the particulate fraction and are thus partially removed by mechanical separation methods.

Pancreatin represents an exemplary biological extract which contains solids owing to its natural virus content and its suspension-like character. PPV is a frequently used model virus, as it is characterised by a very high resistance to a wide variety of inactivation methods. A method which is capable of significantly inactivating PPV also generally leads to high depletion factors for other viruses. In classical spiking experiments, the corresponding substance is spiked with laboratory strains of PPV. These sometimes behave differently from the wild-type strains present in pancreatin.

US-A-2007/0003430 discloses a method for inactivating micro-organisms and viruses, in which the irradiation of a biological liquid is provided with UV radiation. The method provides the use of a special reactor, through which the biological liquid is guided. The method requires the use of a primary flow and a secondary flow.

EP 1 464 342 discloses a further method for sterilising liquid media by means of UV radiation. This method is also aimed at the irradiation of liquid media.

U.S. Pat. No. 6,749,851 B2 describes a method for sterilising digestive enzyme preparations. This method provides stabilisation of the enzyme preparations before irradiation. To this end, the temperature of the enzyme preparation is lowered to a temperature below the ambient temperature. Exemplary temperatures are 0° C. or below. Pancreas enzymes are specified as the digestive enzyme preparations. Described radiation types include corpuscular radiation such as neutron, electron or proton radiation and electromagnetic radiation such as radio waves, visible and invisible light, IR radiation, UV radiation, x-ray radiation and g radiation.

The irradiation of pancreatin preparations which comprise a plurality of enzymes is not shown in the examples. Rather, only the irradiation of liquid or lyophilised trypsin is described, which is carried out with radiation doses of 45 kGy g radiation at a temperature of 4° C. or at room temperature while using a stabiliser. The stabiliser is generally sodium ascorbate, which is used in some cases as a mixture with other stabilisers. The lyophilised samples are resuspended in water before irradiation.

The irradiation of a suspended extract in which some of the enzymes are in solution, whereas some are present bound to solids, is not disclosed.

In Quehl et al. (Die Nahrung 29 (1985) 1, 105-107), the degermination of pancreatin preparations by means of gamma rays is described. In this case radiation doses of 5 to 15 kGy from a ⁶⁰Co source were used. The results show that a radiation dose of 7.5 kGy is sufficient to inactivate all the living germs. However, the loss of activity of the lipases at this radiation dose is between 15 and 20%. At a lower dose of 5.0 kGy, an incomplete inactivation of living germs is achieved, whereas at higher radiation doses (10.0 and 15.0 kGy) the lipase activity is reduced further. At 15 kGy it is only 66% after treatment. These results should correspond with Schlatter et al. (Diss. Nr. 4757, ETH Zürich 1971). The origin of the pancreatin preparation is not described, so that Quehl et al. do not indicate in which form the preparation was present.

DE-OS-25 12 746 discloses a method for producing low-germ, filament-free pancreatin. In this method, irradiation of the pancreatin is not provided. In the description of the prior art in that document it is stated that irradiation of pancreatin with γ rays is known from Schlatter et al. The disclosure content of DE-OS-25 12 746 thus does not proceed from Quehl et al. but confirms the correctness of the statement that irradiation is associated with a loss in enzymic activity.

The method which is provided in DE-OS-25 12 746 achieves a reduction in the germ content by the action of mixtures of liquid chlorinated hydrocarbons and liquid fluorinated hydrocarbons on frozen, extremely finely comminuted pancreas glands. The hydrocarbons at the same time ensure degreasing of the preparation, which is asserted to be a particular advantage. After the fat/solvent phase has been separated off, the enzyme phase is concentrated and dried.

DE-OS-2 135 025 discloses a method for producing a low-germ, fluid pancreatin preparation and a method for its production. The method is based on the finding that the ferment system is at least partially destroyed, that is, the enzymic activity is reduced, by irradiation. It is therefore proposed to process the pancreatin preparation with an aqueous solution or an emulsion of a low aliphatic ketone to form a plastic mass, and then to comminute and dry the latter.

There is furthermore a demand for methods in which the viral and bacterial content in a biological extract which contains solids is reduced completely or to a minimum. The method must be equally suitable for solids and suspensions. The method should in particular make possible the inactivation of PPV strains in pancreatin.

### OBJECT, SOLUTION, ADVANTAGE

The object of the invention is to eliminate the disadvantages of the prior art. In particular, a method for reducing the virus and micro-organism content in biological extracts which contain solids is to be specified, which is suitable for solids and suspensions, does not substantially reduce the activity of the enzymes contained in the biological extract, does not impair the pharmacologically intended properties and does not produce any toxic chemical compounds. Furthermore, products produced by means of the method and uses of the products should be specified.

It is furthermore the object of the invention to provide an extract for the production of pharmaceutical therapeutic agents and for use as foodstuff or nutritional supplement, with the activity of the enzymes contained in the extract not being substantially reduced, and the pharmacologically intended properties not being impaired, and no toxic chemical compounds being produced.

This object is achieved by the features of Claims 1 and 19. Expedient configurations of the invention can be found in the features of the further claims.

According to the invention a method for reducing the virus and micro-organism content of biological extracts which contain solids is provided, comprising
(a) the provision of a biological extract which contains solids in the form of a suspension, which consists of a liquid phase and solid particles dispersed therein, wherein the extract in step (a) comprises a mixture of enzymes, proteins and peptides, some of which is dissolved in the liquid phase and some of which can be bound to the solid particles or is present in pulverulent, solid form; and
(b) the irradiation of the biological extract provided in step (a);
   wherein
   the radiation used for the irradiation is selected from the group comprising ultra-violet radiation, x-ray radiation, β radiation and γ radiation; and
   the enzymic activity of the biological extract which contains solids after irradiation is at least 50% of the enzymic activity of the biological extract which contains solids before irradiation.

The term “biological extract which contains solids” in this case means an extract which (i) preferably contains a mixture of enzymes, proteins and peptides, which (ii) has been obtained as an alcoholic or aqueous extract from animal organs (pancreas, liver, gastric mucosa) and (iii) can be present both in solution and bound to solids. A biological extract which contains solids of this type is a complex extract. The enzyme, protein and peptide mixture preferably has pharmaceutical activity.

An example of such an organ extract is pancreatin, which is obtained from the pancreas, in particular from the pancreas of pigs. Pancreatin is obtained as a pharmaceutical active agent from the pancreas. Pancreatin contains the enzymes lipase, amylase and protease, among others. Pancreatin is thus a biological extract which contains solids in the sense of the present invention. Pancreatin is preferably provided as an aqueous-alcoholic extract in step (a).

The method according to the invention is applicable to all virus forms, in particular DNA and RNA viruses, enveloped and unenveloped viruses, furthermore to virions and prions or similar biological systems and bacteria. The method is preferably used for reducing the PPV content in pancreatin from the pig pancreas.

The method according to the invention allows the reduction of the virus and micro-organism content of biological extracts which contain solids without its enzymic activity being substantially reduced, the pharmacologically intended properties being impaired or toxic chemical compounds being produced. Furthermore, products produced by means of the method and uses of the products should be specified.

The enzymic activity of the biological extract which contains solids after irradiation should be at least 50% of the enzymic activity of the biological extract which contains solids before irradiation, preferably at least 80%, particularly preferably at least 90%.

The viral infectivity of the biological extract which contains solids after irradiation should be reduced by a factor of more than 1 log₁₀ compared to the viral infectivity before irradiation. This applies in particular to the viral infectivity of porcine parvovirus (PPV). For example, the PPV infectivity in pancreatin after treatment should be reduced by a factor of more than 1 log₁₀, preferably more than 3 log₁₀, particularly preferably more than 4 log₁₀, compared to its infectivity before treatment. The germ content after irradiation is preferably no more than 500 CFU/g, preferably no more than 100.

No chemical substances are added during the irradiation of the extract, so that the content of toxic substance after treatment is no higher than before treatment.

Step (b) is preferably carried out using β or γ radiation and x-ray or UV radiation (in particular also UV C radiation) of different wavelengths or intensity. According to the invention, the irradiation of the solid pulverulent extract, of the extract suspended in different solvents or dilutions of these suspensions is provided. Accordingly, the extract provided in step (a) can be suspended in a solvent before step (b). In the case of pancreatin the solvent is preferably a water/alcohol mixture, particularly preferably a water/isopropanol mixture. The dilution preferably takes place at a ratio of 1:1 (extract:solvent) to 1:20, preferably 1:1 to 1:10, particularly preferably 1:1 to 1:5.

The irradiation can take place in a continuous method or in a batch method in a container with stirring. A uniform irradiation of the extract can be achieved with liquids which contain solids by varying the layer thickness (continuous method) or the stirrer speed (batch method). With solid samples, uniform irradiation can be achieved by variation of the layer thickness or suitable swirling. The exposure time of the irradiation on the extract provided in step (a) is preferably more than 15 min. The irradiation is preferably carried out at temperatures between −10° C. and 40° C., particularly preferably between 2° C. and 30° C. and even more preferably at 20° C.

According to the invention, a biological extract which contains solids is furthermore provided with the features of Claims 19 to 26, which was obtained by the method according to the invention. This extract is characterised by a low viral and bacterial content. Despite the previous irradiation, its enzymic activity is not substantially reduced, its pharmacologically intended properties are not impaired and it has no toxic chemical compounds which have arisen owing to the irradiation.

The extract according to the invention which is obtained by irradiation can be used for the production of pharmaceutical therapeutic agents, in particular in the context of oral enzyme therapy, and as a foodstuff or nutritional supplement. These uses are in particular characterised in that the biological extract which contains solids has been obtained by
(a) the provision of a biological extract which contains solids; and
(b) the irradiation of the biological extract provided in step (a);
   wherein
   the radiation used for the irradiation is selected from the group comprising ultra-violet radiation, x-ray radiation, β radiation and γ radiation; and
   the enzymic activity of the biological extract which contains solids after irradiation is at least 50% of the enzymic activity of the biological extract which contains solids before irradiation.

### SHORT DESCRIPTION OF THE DRAWING

The invention is explained in more detail below using examples, which are not intended to limit the invention, with reference to the drawings. In the drawings,

FIG. 1 shows a diagram which shows the reduction of the M2 phage titers during UV C irradiation according to the invention of undiluted extract and extract which has been diluted 1:10;

FIG. 2 shows a diagram which shows the relative enzymic activities after treatment of pancreatin with different radiation doses in percent, based on the starting activity of the untreated sample; and

FIG. 3 shows a diagram which shows the relative enzymic activity and germ count after treatment of pancreatin with different radiation doses. The enzyme activities in percent relate to the starting activities of the untreated sample. The lipase activity has been determined to check the stability after three and six months.

### DETAILED DESCRIPTION OF THE INVENTION AND BEST WAYS OF IMPLEMENTING THE INVENTION

### Examples

### Example 1

### UV Irradiation of a Biological Extract which Contains Solids

The following example describes the treatment of pancreatin as a biological extract which contains solids and has been obtained from pig pancreas, with UV C radiation.

### (1) Method

The experiments were carried out in a continuous throughput reactor. An intermediate stage, which was dissolved in 40% isopropanol, of the pancreatin production process was treated with UV C radiation (254 nm) at 2 J/cm². This dose is normally sufficient for an effective inactivation of parvoviruses. An M2 phage was used as a model for PPV, which M2 phage is characterised like PPV by a very small genome. As the inactivation by UV irradiation is based on damage to the nucleic acids, a comparable genome size is particularly important for the transferability of the results. Alternatively, the same source material was exposed to UV C irradiation over a period of a plurality of hours in a batch stirring reactor and the kinetics of the phage inactivation were recorded. The same experiment was repeated with material diluted 1:10.

### (2) Results

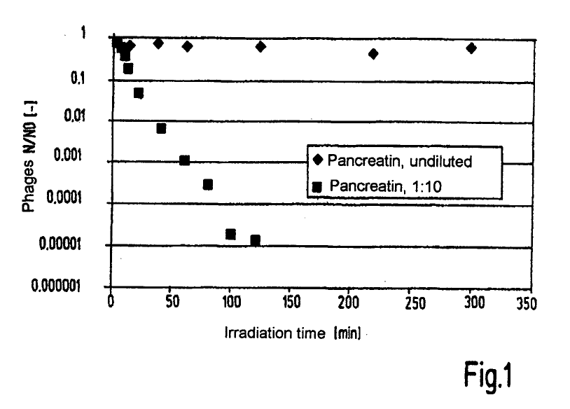

The reduction of the phage titer in the stirring reactor for undiluted and diluted pancreatin as a function of the incubation period is shown in FIG. 1.

### (3) Evaluation

The presented results show that the lack of inactivity of M2 phages in undiluted source material can probably be attributed to a high concentration of solids. Under the selected conditions (high protein, DNA, fat and solid content), a large amount of the UV light is absorbed by the pancreatin components present. Significant depletion kinetics for the M2 phages owing to damage to the DNA could be detected only with use of a 1:10 dilution.

### Example 2

### γ Irradiation of a Biological Extract which Contains Solids

The following example describes the treatment of pancreatin as a solid biological extract which has been obtained from pig pancreas, with γ radiation.

### (1) Method

Pancreatin (active agent) was irradiated in doses of from 1 to 27 kGy and then checked for remaining enzymic activities. After a dose of 5 kGy, additional measurements of enzyme activity after 10 months (stability) and a determination of the CFU/g (germ content) were carried out.

### (2) Results

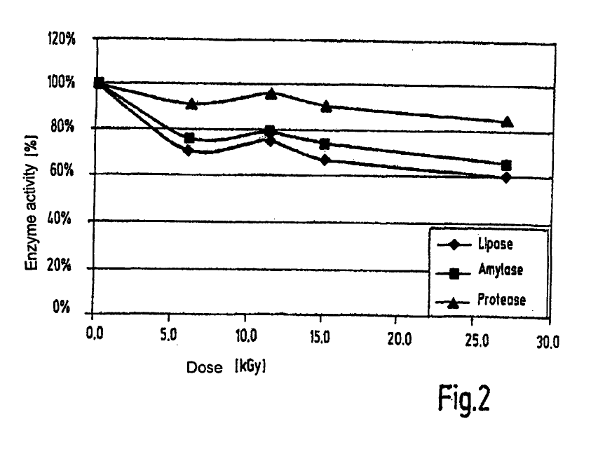

The relative enzymic activities after treatment of pancreatin with different radiation doses in percent, based on the starting activity of the untreated sample are shown in FIG. 2. The stability of the pancreatic enzymes and the reduction of the germ content are shown in Table 1.

TABLE 1
Stability of pancreatic enzymes and reduction of
germ content after γ irradiation (5 kGy)

| | Lipase | Amylase | Protease | CFU/g |
|---|---|---|---|---|
| | activity % | activity % | activity % | % |
| Before irradiation | 100 | 100 | 100 | 47000 |
| After irradiation | 87 | 95 | 100 | 120 |
| After 10 months | 80 | 86 | 97 | 80 |

### (3) Evaluation

At the maximum radiation dose used for the investigations (27 kGy), a maximum reduction in activity (lipase) of approx. 40% is produced. The question of whether this radiation dose is necessary for an effective inactivation of parvoviruses cannot currently be answered. A substantially lower dose of 5 kGy is however sufficient to reduce the bacteria content by more than 2.5 log₁₀ stages. The loss of enzymic activity was only 13% (lipase) under these conditions.

### Example 3

### β Irradiation of a Biological Extract which Contains Solids

The following example describes the treatment of pancreatin as a biological extract which contains solids and has been obtained from pig pancreas, with β radiation.

### (1) Method

Pancreatin (active agent) was irradiated in doses of from 4 to 20 kGy and then checked for remaining enzymic activities. The lipolytic activity was checked again after 3 and 6 months in storage.

### (2) Results

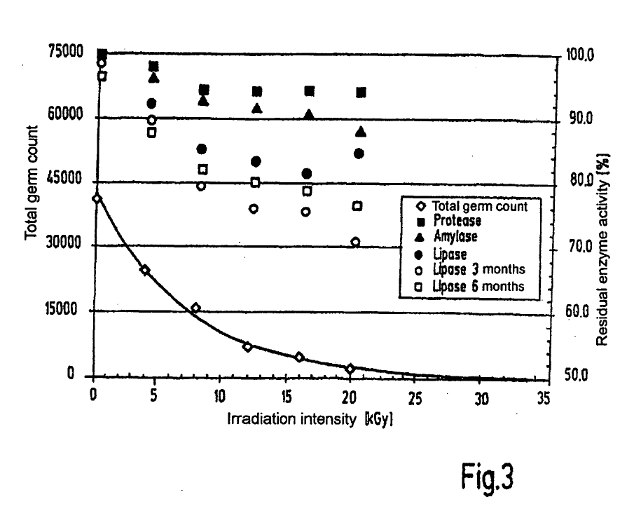

The relative enzymic activity and germ count after treatment of pancreatin with different radiation doses is shown in FIG. 3. The enzyme activities in percent relate to the starting activities of the untreated sample. The lipase activity has been determined to check the stability after three and six months.

### (3) Evaluation

When pancreatin was treated with β radiation, residual activities of >85% could be measured for all investigated enzymes even at a radiation dose of 20 kGy. At the same time the germ count was reduced by approx. 1.5 log₁₀ stages. Despite losses in stability, β radiation is thus a suitable method for reducing the content of micro-organisms in pancreatin.

1. Method for reducing the virus and micro-organism content of biological extracts which contain solids, comprising
(a) the provision of a biological extract which contains solids in the form of a suspension, which consists of a liquid phase and solid particles dispersed therein, wherein the extract in step (a) comprises a mixture of enzymes, proteins and peptides, some of which is dissolved in the liquid phase and some of which is bound to the solid particles or is present in solid, pulverulent form; and
(b) the irradiation of the biological extract provided in step (a);
wherein
the radiation used for the irradiation is selected from the group comprising ultra-violet radiation, x-ray radiation, β radiation and γ radiation; and
the enzymic activity of the biological extract which contains solids after irradiation is at least 50% of the enzymic activity of the biological extract which contains solids before irradiation.

2. Method according to claim 1, wherein
the biological extract which contains solids has been obtained from an animal organ.

3. Method according to claim 1, wherein
the biological extract which contains solids is an extract from the pancreas of a pig.

4. Method according to claim 1, wherein
the enzymic activity of the biological extract which contains solids after irradiation is at least 50% of the enzymic activity of the biological extract which contains solids before irradiation.

5. Method according to claim 1, wherein
the enzymic activity of the biological extract which contains solids after irradiation is at least 80% of the enzymic activity of the biological extract which contains solids before irradiation.

6. Method according to claim 1, wherein
the enzymic activity of the biological extract which contains solids after irradiation is at least 90% of the enzymic activity of the biological extract which contains solids before irradiation.

7. Method according to claim 1, wherein
the viral infectivity of the biological extract which contains solids after irradiation has been reduced by a factor of more than 1 log₁₀ compared to the viral infectivity before irradiation.

8. Method according to claim 1, wherein
the viral infectivity of the biological extract which contains solids after irradiation has been reduced by a factor of more than 3 log₁₀ compared to the viral infectivity before irradiation.

9. Method according to claim 1, wherein
the viral infectivity of the biological extract which contains solids after irradiation has been reduced by a factor of more than 4 log₁₀ compared to the viral infectivity before irradiation.

10. Method according to claim 1, wherein
the viral infectivity of porcine parvovirus of the biological extract which contains solids after irradiation has been reduced by a factor of more than 1 log₁₀ compared to the viral infectivity of porcine parvovirus before irradiation.

11. Method according to claim 1, wherein
the viral infectivity of porcine parvovirus of the biological extract which contains solids after irradiation has been reduced by a factor of more than 3 log₁₀ compared to the viral infectivity of porcine parvovirus before irradiation.

12. Method according to claim 1, wherein
the viral infectivity of porcine parvovirus of the biological extract which contains solids after irradiation has been reduced by a factor of more than 4 log₁₀ compared to the viral infectivity of porcine parvovirus before irradiation.

13. Method according to claim 1, wherein
the content of toxic substances in the biological extract which contains solids is no higher after irradiation than before irradiation.

14. Method according to claim 1, wherein
the germ content is less than 500 CFU/g after irradiation.

15. Method according to claim 1, wherein
the germ content is less than 100 CFU/g after irradiation.

16. Method according to claim 1, wherein
the process temperature in step (b) is between −10° C. and 40° C.

17. Method according to claim 1, wherein
the process temperature in step (b) is between 2° C. and 30° C.

18. Method according to claim 1, wherein
the process temperature in step (b) is 20° C.

19. Biological extract which contains solids and has reduced viruses and micro-organisms for the production of pharmaceutical therapeutic agents for use as a foodstuff or nutritional supplement, obtained by the method according to claim 1.

20. Extract according to claim 19,
wherein the extract is obtained from an animal organ, the extract is preferably an extract from the pancreas of a pig.

21. Extract according to claim 19, wherein
the enzymic activity of the extract after irradiation
a.) is at least 50% of the enzymic activity of the extract before irradiation or
b.) is at least 80% of the enzymic activity of the extract before irradiation or
c.) is at least 90% of the enzymic activity of the extract before irradiation.

22. Extract according to claim 1, wherein
the viral infectivity of the extract after irradiation
a.) is reduced by a factor of more than 1 log₁₀ compared to the viral infectivity before irradiation or
b.) is reduced by a factor of more than 3 log₁₀ compared to the viral infectivity before irradiation or
c.) is reduced by a factor of more than 4 log₁₀ compared to the viral infectivity before irradiation.

23. Extract according claim 19, wherein
the viral infectivity of porcine parvovirus of the extract after irradiation
a.) is reduced by a factor of more than 1 log₁₀ compared to the viral infectivity of porcine parvovirus before irradiation or
b.) is reduced by a factor of more than 3 log₁₀ compared to the viral infectivity of porcine parvovirus before irradiation or
c.) is reduced by a factor of more than 4 log₁₀ compared to the viral infectivity of porcine parvovirus before irradiation.

24. Extract according to claim 19, wherein
the content of toxic substances in the extract is no higher after irradiation than before irradiation.

25. Extract according to claim 19, wherein
the germ content after irradiation
a.) is less than 500 CFU/g or
b.) is less than 100 CFU/g.

26. Extract according to claim 19, wherein
the process temperature in step (b)
a.) is between −10° C. and 40° C. or
b.) is between 2° C. and 30° C. or
c.) is 20° C.

27. Use of a biological extract which contains solids according to claim 19, for producing a medicament or as a foodstuff or nutritional supplement.

28. Use according to claim 27, wherein
the biological extract which contains solids has been obtained by
(a) the provision of a biological extract which contains solids; and
(b) the irradiation of the biological extract provided in step (a);
wherein
the radiation used for the irradiation is selected from the group comprising ultra-violet radiation, x-ray radiation, β radiation and γ radiation; and
the enzymic activity of the biological extract which contains solids after irradiation is at least 50% of the enzymic activity of the biological extract which contains solids before irradiation.

29. Use according to claim 27, wherein
the biological extract which contains solids which is obtained after irradiation is used for producing a medicament.

30. Use according to claim 27, wherein
the biological extract which contains solids which is obtained after irradiation is used as a foodstuff or nutritional supplement.
